(19) United States
(12) Patent Application Publication
(10) Pub. No.: US 2020/0298060 A1
(43) Pub. Date: Sep. 24, 2020

(54) SYSTEM AND METHOD FOR THE MEASURE OF IMPACT KINETICS

(71) Applicant: Zev Industries Ltd., Coquitlam (CA)

(72) Inventors: Seth Mathew Lenetsky, Victoria (CA); Roy J. Nates, Auckland (NZ); Steven Douglas Graham, Coquitlam (CA)

(73) Assignee: Zev Industries Ltd., Coquitlam (CA)

(21) Appl. No.: 16/572,539

(22) Filed: Sep. 16, 2019

Related U.S. Application Data
(60) Provisional application No. 62/822,230, filed on Mar. 22, 2019.

(51) Int. Cl. A63B 24/00

(52) U.S. Cl. CPC A63B 24/0062; A63B 2225/50; A63B 2220/833; A61B 2562/0219; A63B 2220/53; A63B 2220/40

(57) ABSTRACT

Provided is a system and method for measuring impact kinetics. The system includes a strike target having an outer surface with at least one pressure sensor proximate thereto, a known center of gravity and a known weight, the strike target further having a pivot attachment to an overhead support. At least one accelerometer is associated with the strike target as well. The system also includes a detection system in communication with the at least one pressure sensor and the accelerometer, the detection system initiating kinetic impact determination upon a signal to evaluate a strike, the strike producing strike data including at least a moment of the bag around the pivot attachment, this moment causing an angular acceleration (\vec{α}) of the strike target around the pivot as detected by the at least one accelerometer and reported to the detection system; wherein the detection system determines impact kinetics as a natural frequency (ωₙ) and a moment of inertia (I_{P}) of the strike target as a compound pendulum, determines the circular frequency (−ωₙ) of the compound pendulum, and a resulting impact force as a function of instantaneous acceleration, the impact kinetics reported to a user. An associated method of use is also provided.

### CROSS REFERENCE TO RELATED APPLICATIONS

This application claims the benefit under 35 U.S.C. § 119(e) of U.S. Provisional Application No. 62/822,230 filed Mar. 22, 2019 and entitled SYSTEM AND METHOD FOR THE MEASUREMENT OF IMPACT KINETICS, the disclosure of which is incorporated herein by reference.

### FIELD OF THE INVENTION

The present invention relates generally to the measurement of impact kinetics produced by striking impacts of combat sport athletes.

### BACKGROUND

The measurement of striking (punches, kicks, elbows and knees) is generally known as impact kinetics, and has been explored substantially in combat sports literature.

This area has been identified in combat sports related literature as an important variable due to the key role impact has in the performance of full contact combat sports. A variety of devices have been used in the literature and developed commercially, but none realistically reflect the inertial characteristics of human targets and training equipment. More specifically, they fail to report data in validated scientific units. In addition, the systems that do exist utilize highly specific input and output devices and are rather expensive, failing to recognize that mobile devices are widely present and already familiar to users.

There are discrepancies in the literature regarding the specific factors (muscle activation patterns, the role of the lower body, and ideal training stimulus) that result in effective impact kinetics. Still, it has been recognized that, in general, impact kinetics play a key role in the performance of full contact combat sports athletes.

The use of striking dynamometers on immovable (or very rigid) surfaces or objects, commonly seen, fails to reflect the inertial characteristics of a human target and produces results that are specific to striking very rigid targets only. The movement of a rigid target is negligible and therefore the time of contact is very small. This translates poorly to real world predication, as a human target (or training bag) will move significantly during contact, meaning that there is much more time available during the contact.

Moreover, this extended duration of contact will produce very different force profiles between the two that is not accurately accounted for, or otherwise measured by present striking dynamometers or immovable surfaces or other target objects fitted with a traditional impact sensor.

Also, the technique of the striking action would be quite different as a result of a highly rigid target. To avoid introducing such potential error some researchers have used load cells attached to inertially relevant pendulums (i.e. similar mass to a human or human segment), while others have altered tools used by combat sports athletes regularly such as punching bags or human analogues to measure striking kinetics.

The scientific units that have been identified as paramount to the measurement of striking kinetics are those related to force. Specifically, Newtons—the force needed to accelerate one kilogram of mass at the rate of one meter per second squared in the direction of the applied force and Impulse—the integration of force over time. That is, a vector quantity which produces equivalent vector change in the linear momentum, also in the same direction.

In the literature, peak force has most commonly been reported of all variables. Impulse conversely, has not been found to be reported. This, despite the theoretical importance given to Impulse. In commercial products the most commonly reported values are “Intensity” an arbitrary unit that claims to reflect striking force but lacks any scientific foundation or a “G” result that measures the changes in the acceleration of the hand, not the impact target. Of the few devices that report in scientific units, Impulse has not been found reported.

The methods of measuring impact kinetics vary throughout the literature. The most common designs include: force plates or load cells, fluid filled targets, strain gauges, and accelerometers inserted internally in strike targets. These dynamometers typically require expensive and complicated equipment (that fail to integrate with the modern mobile device environment) to achieve measurement, with acceptable reliability and validity.

Commercially, products used to measure impact kinetics have fallen into three categories: 1) inertially irrelevant targets that produce scientific units (Loadstar and PowerKube), 2) inertially relevant targets that are unable to produce scientific units (UFC Force Tracker and ImpactWrap), and 3) athlete worn measurement devices that are unable to produce scientific units (Everlast PIQ, Corner, Strike Tec, Hyskso, and Jaboo). Although some of the devices in this third category do connect to mobile devices, this ease of access doesn't improve the quality or usefulness of their data, and such wireless connection ability is actually missing in those devices from categories 1 and 2 which strive to capture better data.

Hence, the there is a need for a system and method of determining impact kinetics that overcomes one or more of the above identified challenges.

### SUMMARY OF THE INVENTION

Our invention solves the problems of the prior art by providing novel systems and methods for measurement and/or determination of impact kinetics.

In particular, and by way of example only, according to one embodiment of the present invention, provided is a system for measuring impact kinetics, including: a strike target having an outer surface with at least one pressure sensor proximate thereto, a known center of gravity and a known weight, the strike target further having a pivot attachment to an overhead support; at least one accelerometer associated with the strike target; a detection system in communication with the at least one pressure sensor and the accelerometer, the detection system initiating kinetic impact determination upon a signal to evaluate a strike, the strike producing strike data including at least a moment of the bag around the pivot attachment, this moment causing an angular acceleration (\vec{α}) of the strike target around the pivot as detected by the at least one accelerometer and reported to the detection system; wherein the detection system determines impact kinetics as a natural frequency (ωₙ) and a moment of inertia (I_{P}) of the strike target as a compound pendulum, determines the circular frequency (−ωₙ) of the compound pendulum, and a resulting impact force as a function of instantaneous acceleration, the impact kinetics reported to a user.

For another embodiment, provided is a method for measuring impact kinetics, including: providing a strike target having a pivot attachment to an overhead support, an outer surface with at least one pressure sensor proximate thereto, and a known center of gravity, and weight of the bag, the strike target having at least one accelerometer associated therewith; providing a detection system in communication with the at least one pressure sensor and the accelerometer; and initiating kinetic impact determination upon a signal to evaluate a strike, the strike producing strike data including at least a moment of the bag around the pivot attachment, this moment causing an angular acceleration (\vec{α}) of the target around the pivot as detected by the at least one accelerometer and reported to the detection system; wherein the detection system determines impact kinetics as a natural frequency (ωₙ) and a moment of inertia (I_{P}) of the strike target as a compound pendulum, determines the circular frequency (−ωₙ) of the compound pendulum, and a resulting impact force as a function of instantaneous acceleration, the impact kinetics reported to a user.

And still, for yet another embodiment, provided is a system for measuring impact kinetics, including: A strike detection system having at least one processor and adapted to receive strike data from a strike target, the strike data provided by at least one pressure sensor and at least one accelerometer associated with the strike target having a known center of gravity and a known weight, the strike target further having a pivot attachment to an overhead support; at least one remote strike detection controller having at least one processor and non-volatile memory coupled to the processor having processor executable instructions to direct operation of strike detection system, the strike detection controller having a wireless network component coupled to the processor and the non-volatile memory and in communication with the strike detection system; a strike evaluator for initiating kinetic impact determination upon a signal to evaluate a strike, the strike producing the strike data as a moment of the bag around the pivot attachment, this moment causing an angular acceleration ((\vec{α}) ) of the strike target around the pivot as detected by the at least one accelerometer and reported to the detection system; wherein the strike evaluator determines impact kinetics as a natural frequency (ωₙ) and a moment of inertia (I_{P}) of the strike target as a compound pendulum, determines the circular frequency (−ωₙ) of the compound pendulum, and a resulting impact force as a function of instantaneous acceleration, the impact kinetics reported to a user.

### BRIEF DESCRIPTION OF THE DRAWINGS

FIG. 1 illustrates a high level diagram of a system for measuring impact kinetics in accordance with at least one embodiment;

FIG. 2 is a schematic block diagram of the data process and methodology of at least one embodiment;

FIG. 3 is a flow diagram for measuring impact kinetics in accordance with at least one embodiment;

FIGS. 4A and 4B are enlarged conceptual illustrations of a strike target at rest FIG. 4A, and when struck FIG. 4B showing key elements and relationships as utilized in equations for measuring impact kinetics in accordance with at least one embodiment;

FIG. 5 presents raw acceleration graphs for the X-Axis, Y-Axis and Z-Axis during the measuring of impact kinetics in accordance with at least one embodiment;

FIG. 6 presents the raw force curve calculation based on the raw acceleration data of FIG. 5, the final kinetic output variables of peak force and Impulse, an exemplary screen shot from a portable computing device such as a smart phone demonstrating how the final kinetic variables are presented to a user in accordance with at least one embodiment;

FIG. 7 a high level block diagram of a computer system in accordance with at least one embodiment.

### DETAILED DESCRIPTION

Before proceeding with the detailed description, it is to be appreciated that the present teaching is by way of example only, not by limitation. The concepts herein are not limited to use or application with a specific system or method for measuring striking impacts. Thus, although the instrumentalities described herein are for the convenience of explanation shown and described with respect to exemplary embodiments, it will be understood and appreciated that the principles herein may be applied equally in other types of systems and methods involving the measurement of striking impacts.

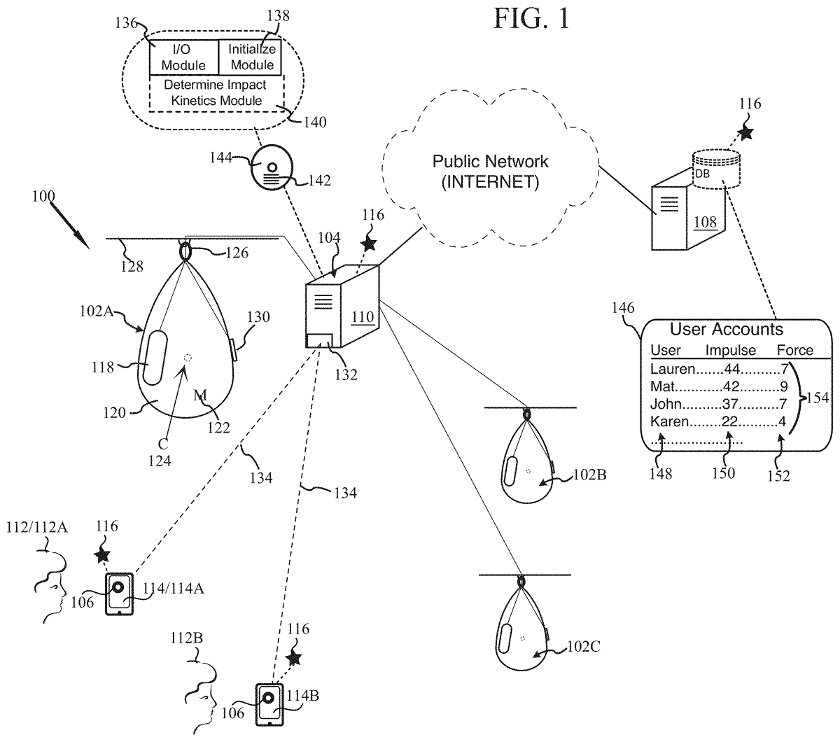

This invention is described with respect to preferred embodiments in the following description with reference to the Figures, in which like numbers represent the same or similar elements. Further, with the respect to the numbering of the same or similar elements, it will be appreciated that the leading values identify the Figure in which the element is first identified and described, e.g., element 100 first appears in FIG. 1.

To briefly summarize, provided is a system and method that permits the measurement of striking impacts, hereinafter impact connect. This is achieved by the measurement of acceleration after a strike target (an inertially relevant target) is hit. In varying embodiments, this strike target may be a tear drop bag, ball, or small punching bag.

Various embodiments presented herein are descriptive of apparatus, systems, articles of manufacturer, or the like for systems and methods involving determining impact kinetics. In some embodiments, an interface, application browser, window or the like may be provided that allows the user of the computing device to direct behavior of the computing device.

Moreover, some portions of the detailed description that follows are presented in terms of the manipulation and processing of data bits within a computer memory. The steps involved with such manipulation are those requiring the manipulation of physical quantities. Generally, though not necessarily, these quantities take the form of electrical or magnetic signals capable of being stored, transferred, combined, compared and otherwise manipulated. Those skilled in the art will appreciate that these signals are commonly referred to as bits, values, element numbers or other clearly identifiable components.

It is of course understood and appreciated that all of these terms are associated with appropriate physical quantities and are merely convenient labels applied to these physical quantifies. Moreover, it is appreciated that throughout the following description, the use of terms such as “processing” or “evaluating” or “receiving” or “outputting” or the like, refer to the action and processor of a computer system or similar electronic computing device that manipulates and transforms the data represented as physical (electrical) quantities within the computer system's memories into other data similarly represented as physical quantities within the computer system's memories.

The present invention also relates to an apparatus for performing the operations herein described. This apparatus may be specifically constructed for the required purposes as are further described below, or the apparatus may be a general purpose computer selectively adapted or reconfigured by one or more computer programs stored in the computer upon computer readable storage medium suitable for storing electronic instructions.

Turning now to FIG. 1 there is shown an embodiment for an Impact Kinetics Measurement System (“IKMS”) 100 in accordance with at least one embodiment. As is further described below, IKMS 100 is structured and arranged to advantageously permit and achieve the measuring of striking impacts, hereinafter referred to as impact kinetics. This is achieved by the measurement of acceleration after a strike target (an inertially relevant target) is hit.

As shown in FIG. 1, for at least one embodiment, IKMS 100 includes a strike target 102, a detection system 104, a remote application 106, and optionally a remote database 108. As shown, for at least one embodiment, the detection system 104 is provided by at least one physical computer system 110 (including at least one microprocessor, memory, I/O device(s) and the like) that is adapted by hardware or software to provide detection system 104. For embodiments of IKMS 100 which include a remote database and/or computing system 108, such as cloud based storage and computing recourses (e.g., cloud resources), the cloud resources are understood and appreciated to be provided by one or more physical computing system.

For at least one embodiment, IKMS 100 is advantageously operable with a plurality of users 112, of which users 112A and 112B are exemplary, and a plurality of strike targets 102, of which strike targets 102A, 102B and 102C are exemplary.

For one exemplary embodiment, interaction with IKMS 100 by users 112 is facilitated by each user 112 having a portable computing device, e.g., first device 114, such as but not limited to a smart phones or tablet devices, such as but not limited to iPhone®, iPad®, Android® or other portable computing device, having the remote application 106 installed thereon which adapts the first device 114 for interaction with IKMS 100 as a strike detection controller. For at least one alternative embodiment, IKMS 100 may optionally permit users 112 to connect via a web browser in place of the remote application 106.

IKMS 100 also includes a strike evaluator 116 (shown as a star) for initiating kinetic impact determination upon a signal to evaluate a strike made to the strike target 102. As will be further discussed below, in varying embodiments, the strike evaluator 116 may be a component of the detection system 104, a component of the remote application 106 and thus an element of the first device 114 adapted as a strike detection controller, or a component of the remote database 108. For still yet another embodiment, the strike evaluator 116 may be a distinct and separate adapted computer system operating in communication with other computing elements of IKMS 100. Indeed, multiple instances of strike evaluator 116 may exist within IKMS 100.

Moreover, IKMS 100 may be summarized as a strike detection system 104, a remote strike detection controller (provided by remote application 106) and a strike evaluator 116, cooperatively interacting with each other and a strike target 102.

In varying embodiments, the strike target 102 may be a tear drop bag, ball, or small punching bag. The strike target 102 also has at least one pressure sensor 118, typically disposed on or proximate to the outer surface 120 of the strike target 102, a known mass M 122 and a known center of gravity C 124, and a pivot attachment 126 to an overhead support 128. At least one accelerometer 130 is also associated with the strike target 102. For at least one embodiment, the at least one accelerometer 130 may be disposed on the opposite side of the strike target 102 from the pressure sensor 118. For yet another embodiment, the at least one accelerometer 130 is disposed within the strike target 102.

For yet at least one embodiment, the accelerometer 130 may be an integrated component of detection system 104—and for such embodiments, the detection system 104 is indeed disposed on the opposite side of the strike target 102 from the pressure sensor 118 as shown with respect to FIG. 1 and accelerometer 130. For yet another integrated embodiment, the combined accelerometer 130 and detection system 104 may be at least partially disposed within the strike target 102.

For at least one embodiment the pressure sensor 118 is arranged as a strip having at least 40 sensors horizontally staked in a 100 cm column. The location of each horizontal sensor is predetermined and thus known to IKMS 100. At regular intervals, the pressure sensor 118 is scanned, such as every 100 ms. If a reading has been detected, this data is recognized by the detection system 104 as a strike, and the strike evaluator 116 is engaged to determine the impact kinetics associated with the strike. For at least one embodiment, the pressure sensor 118 is a tactile pressure sensor provided by Sensor Products Inc., of 300 Madison Ave #100, Madison, N.J. 0794 (https://www.sensorprod.com/index.php).

For at least one embodiment the detection system 104 includes a control board understood and appreciated to be an 8/16/32 g 3D accelerometer controller and the accelerometer 130 disposed upon or within the strike target 102 is an 8/16/32 g 3D accelerometer 130. For at least one alternative embodiment, the control board is a 8/16/64 g 3D accelerometer controller and the accelerometer 130 is an 8/16/64 g 3D accelerometer 130. For at least one embodiment, the accelerometer 130 is a KX222 accelerometer provided by Kionix of Ithaca, N.Y. (https://www.kionix.com).

With respect to the present invention, it is to be noted that long punching bags, such as those of classic gym punching bag style that are over a meter and a half in length are not necessarily desirable for the present invention—without modification. Long punching bags do not move in a pendulum fashion. Instead, due their size these bags experience extreme deformation when struck. Such extreme deformation may well mask some of the kinetic impact energy that the present invention seeks to advantageously determine. Moreover, for use with IKMS 100, a strike target is an object that will swing and/or move in a pendulum fashion without extreme deformation.

For at least one embodiment, the acceleration data is calculated with variables derived from the inertial modelling of a standard “tear drop” striking bag as the strike target 102 which produces force variables (peak force and mean force). Inertial modelling is the process of:
locating the strike target 102 pivot point;
locating the strike target 102 center of gravity;
locating the point of impact (measured in a case by case manner via the pressure sensor mounted to the front);
locating the position of the accelerometer(s); and
locating the distances of the above from the pivot point.

For at least one embodiment, the present invention incorporates at least one 3D accelerometer 130, which may also be known or referred to as a triaxial accelerometer. In alternative embodiments, there are at least three accelerometers 130, an x-axis accelerometer, a y-axis accelerometer and a z-axis accelerometer.

The sampling rate of the accelerometer 130 allows for the measurement of acceleration during brief period of impact (30-50 ms) which produces a force curve used to calculate Impulse in addition to the previously mentioned force variables. Of course, in varying embodiments the measurement of impact may occur over a longer or shorter duration as may be desired for different implementations. Impulse is a unique and important variable that is advantageously measured by the present invention.

Impulse has been repeatedly theorized in the literature to result in effective strikes when targeting the head. In many full contact combat sports, a strike to the head that results in rendering the opponent unconscious is highly desirable, and as such the ability of the present invention to determine Impulse is highly advantageous. Conversely, peak force is theorized to primarily produce superficial injuries to the head and face. Furthermore, high Impulse strikes are thought to be capable of damaging soft tissue in the torso and limbs, unlike peak force focused impacts. Moreover, embodiments of IKMS 100 advantageously determine Impulse which may in turn be communicated to users of IKMS 100 in a variety of different ways as further set forth below.

For at least one embodiment, the detection system 104, which may also be considered as a main sensor base station, is an adapted computer system in communication with the pressure sensor 118 and the at least one accelerometer 130 associated with each strike target 102. Indeed, in varying embodiments, IKMS 100 may provide a plurality of strike targets 102 for substantially concurrent use by a plurality of users, and/or a plurality of strike targets 102 arranged for use by a single user 112, e.g. punching targets and kicking targets.

The detection system 104 also includes a wireless network component 132, that provides wireless communication 134 with the first device 114, such as by 802.11 network, or an ad-hoc network, such as but not limited to a Bluetooth connection. As such, detection system 104 thereby provide a user with advantageous data and information regarding the determination of impact kinetics.

Although the communication with the pressure sensor 118 and/or the at least one accelerometer 130 may be wireless, for at least one embodiment physical wiring establishes the communication so as to eliminate the need for the strike target 102 to provide power and wireless communication systems.

Each active instance of the remote application 106 adapts the first device 114 by providing at least an I/O module 136 and an initialize module 138. Optionally, for at least one embodiment, the determination of impact kinetics is substantially performed by the first device 114 as directed by the remote application 106 which also provides a determine kinetics module 140. In varying embodiments, the determine kinetics module 140 may be provided to the detection system 104 which is also provided with an I/O module to communicate with the at least one pressure sensor 118 and the at least one accelerometer 130 of each strike target 102 provided for an embodiment of IKMS 100, as well as to communicate with the remote application 106 operating on each first device 114 and the optional database 108.

It is to be understood and appreciated that each module or system is implemented as a collection of independent electronic circuits packaged as a unit upon a printed circuit board or as a chip attached to a circuit board or other element of a computer so as to provide a basic function within a computer. In varying embodiments, one or more modules may also be implemented as software that adapts a computer to perform a specific task or basic function as part of a greater whole. Further still, in yet other embodiments one or more modules may be provided by a mix of both software and independent electronic circuits.

Moreover, the elements of the remote application 106 (I/O module 136 & Initialize module 138), the detection system 104 and the strike evaluator 116 are conceptually illustrated in the context of an embodiment for at least one computer program 142. Such a computer program 142 may be provided upon a non-transitory computer readable media, such as an optical disc 144 or USB drive (not shown), having encoded thereto an embodiment of a program for IKMS 100.

For at least one embodiment the remote application 106, which may include the strike evaluator 114 as a module, may be provided directly to desiring users 112 by the remote detection system 104. For at least one alternative embodiment, the modules comprising remote application 106 are made available from a third party such as, but not limited to the Apple® App Store, or Google® Play, or such other third party application provider.

For at least one alternative embodiment the determination of impact kinetics is substantially performed by the main sensor base station and reported to the remote application. For yet still another embodiment, as shown IKMS 100 may include a remote database and/or computing system 108, such as cloud based storage and computing recourses (e.g., cloud resources), the cloud resources being adapted by software and/or hardware to perform the calculations for determining the impact kinetics, which are in turn stored with the cloud resources and directed to the remote application.

Moreover, for at least one embodiment the I/O module 136 permits a user to enter personal data so as to create or retrieve a user account with IKMS 100, or at the very least personalize the data recording session for the determination of impact kinetics. Exemplary user account data 146 is shown in FIG. 1, providing the user name 148, and a record of Impulse 150 and force 152. Additional user data fields, such as but not limited to user ID, type of strike (kick/punch/etc.), billing information, user age, sex, weight, and/or other elements may of course be added in various embodiments.

Before proceeding into detailed equations, a high-level overview of the method may be helpful for providing greater context for those not so skilled in the art, but still interested in perceiving the advantages of the present invention. To measure the instantaneous force produced by a strike, baseline modeling of the strike target 102 must be determined while the strike target 102 is at rest. At rest, i.e., the initial state, the 3D accelerometer is oriented to the Earth's gravity so as to determine the initial acceleration of the strike target 102 horizontally—more specifically, the determination of gravity upon the strike target at rest.

This initializes the inertial modeling of the strike target 102 (mass, natural frequency, location of the accelerometer, center of gravity, and distances to the pivot point). Once determined, this initial state or “rest data” is subsequently used for the determination of actual impact kinetics.

Although the initial state may be saved and reused, optionally the initial state is re-determined to ensure that environmental factors have not imparted a change to the strike target 102 which might alter the determination of impact kinetics if not properly accounted for—such as, but not limited to a change in weight due to humidity absorption, the affixation of a doll/picture/tape/or other visual target material, etc. . . .

Moreover, the process of determining the initial baseline values and the kinetic impact values are essentially the same, with the initial baseline values determined when the strike target 102 is at rest, whereas a user's kinetic impact values are determined when the strike target 102 is actually struck.

For at least one embodiment, the user initializes IKMS 100 with a signal from his or her first device 114 as adapted by the remote application 106, specifically the initialize module 138. With the initial baseline so determined, or at least retrieved from a prior recording, for at least one embodiment IKMS 100 indicates to a user that it is ready. For at least one embodiment, this indication is by a sound, color or other visual and/or auditory signal that may be provided from the remote application, the strike target itself, and/or the detection system 104, aka main sensor base station.

In response to the indication of readiness, a user then proceeds to strike the strike target 102. The occurrence of a strike is determined by the pressure sensors 118 and/or accelerometer(s) 130 of the strike target 102. For at least one embodiment, data regarding the pressure as measured by the pressure sensor(s) 118 and motion/movement as determined by the accelerometer(s) 130, “strike data 154 ,” is collected by the detection system 104.

As noted above, in varying embodiments this strike data 154 may be processed by the detection system 104, the remote application 106, and/or by the remote database 108.

More specifically, the strike data is compared with and evaluated to the rest data, and from this comparison and evaluation IKMS 100 determines and outputs one or more data reports regarding the determined impact kinetics, such as Impulse of the strike.

Moreover, the developed equations when combined with the inertial modelling of the strike target 102, and key information regarding the location of the strike allow for the conversion of acceleration in to instantaneous force.

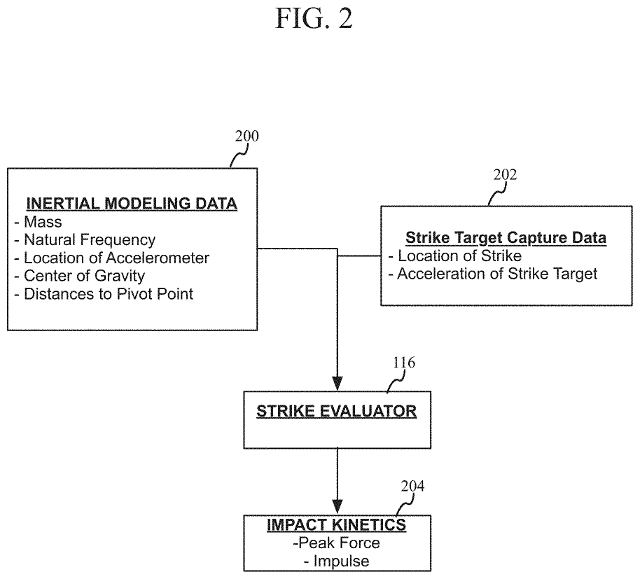

FIG. 2 conceptually illustrates the data process and methodology of at least one embodiment of IKMS 100. More specifically, the collection of modeling data 200—essentially the mass M 122 of the strike target 102, the location of the accelerometer 130, the center of gravity Cg 124, distances between the pressure sensor 118 and Cg 124 to the pivot point 126, and the initial natural frequency of the strike target 102 as a pendulum, and the collection of strike data 202—the location of the strike and the acceleration of the strike target 102, which in turn are combined and processed by the strike evaluator 116 to determine the impact kinetics 204 of at least peak force and Impulse.

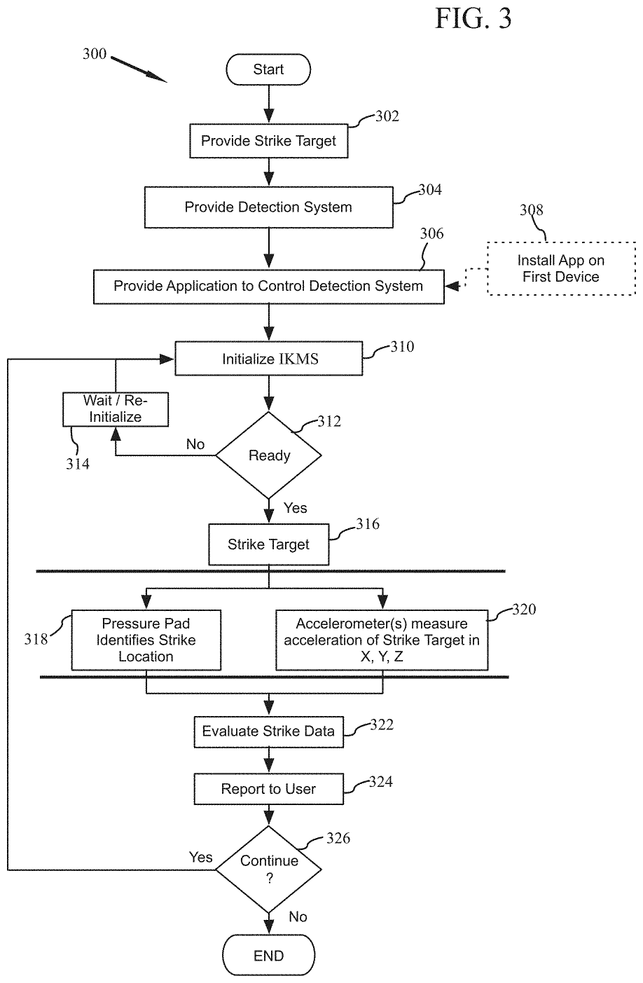

FIG. 3 provides a general flow diagram for a method 300 of determining impact kinetics in accordance with at least one embedment of the present invention. It will be appreciated that the described method need not be performed in the order in which it is herein described, but that this description is merely exemplary of one method of determining impact kinetics in accordance with the present invention.

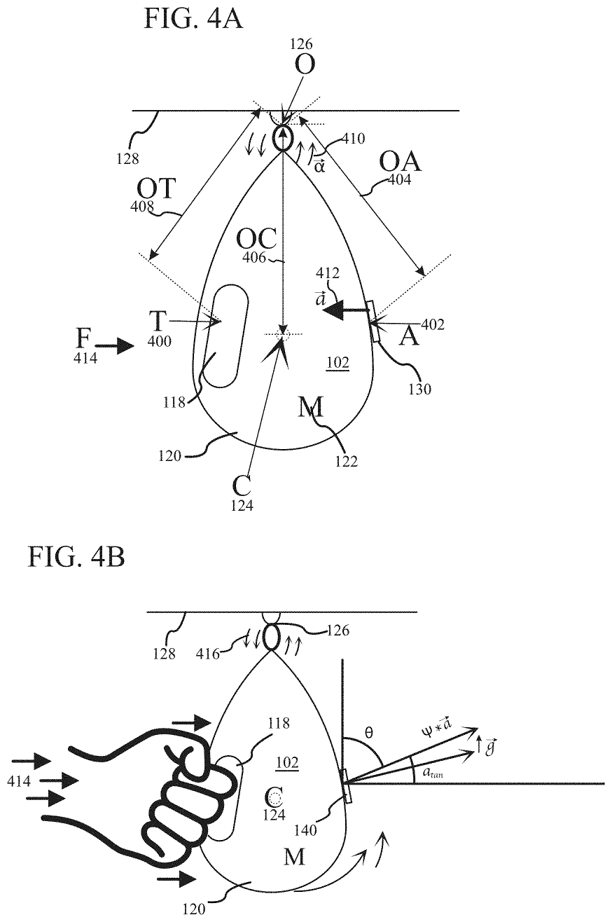

To facilitate the discussion of method 300, in addition to FIG. 1, FIGS. 4A and 4B are presented to illustrate the locations of key points and relationships therebetween. Indeed, for use with the following equations, specific points are identified with letter reference in addition to figure numbers.

In this model the strike target 102 is considered a compound pendulum pivoting around a hanging hinge and the sections of the strike target 102 are identified as follows in FIG. 4A:

O 126 the pivot of the strike target 102.

C 124 the center of gravity of the strike target 102.

T 400 the target striking zone, the point of impact. The target of the strike is identified via a pressure sensor 118 device mounted on the strike target 102.

A 402 the position of the accelerometer 130.

O̅A̅ 404 the distance from the accelerometer to the pivot point (m).

O̅C̅ 406 the distance from the center of gravity to the pivot point (m).

O̅T̅ 408 the distance from the point of impact to the pivot point (m).

The key variables shown in FIG. 4A are:

(\vec{α}) 410 the angular acceleration of the strike target 102 around the pivot 126 in rad/s².

(\vec{a}) 412 the linear acceleration measured at the accelerometer in m/s².

(\vec{F}) 414 the force of the punch thrown by the boxer at the impact point in N (Newtons).

Returning to FIG. 3, for at least one embodiment, method 300 typically begins with establishing the IKMS 100 environment. Moreover, this includes providing or obtaining a strike target 102 as described above, block 302. In addition, the detection system 104 is provided or otherwise obtained, block 304. And further, the user is provided with the remote application 106 to control the detection system 104 of IKMS 100. For at least one embodiment, the user visits an application website from which is downloaded a copy of the remote application 106 which the user then installs on his or her first device, optional block 306.

With the remote application installed and activated, the first device is now adapted at least in part as a remote strike detection controller. The user then initializes IKMS 100 to establish the baseline values, block 310. The user then waits for a Ready indication, decision 312. As noted above, IKMS 100 will indicate a ready state through one of several optional means, such as but not limited to, sound, light, voice recording, and/or combinations thereof. If the user is not certain that IKMS 100 is read, the user may wait, block 314, and possibly re-initialize IKMS 100, block 310.

When ready, the user strikes the strike target, block 316. Upon a strike, the pressure pad identifies the strike location, block 318 and the accelerometer(s) measure the acceleration of the strike target 102 in X, Y and Z coordinate axis, block 320. For at least one embodiment, the determination of location by the pressure pad and the measurement of acceleration by the accelerometer(s) occur substantially simultaneously. This strike data is provided to the detection system which in turn provides the strike data to the strike evaluator. The strike evaluator then compares the strike data to the baseline data to determine the impact kinetics of the strike, block 322. The impact kinetics are then reported to the user, block 324, as well as optionally recorded to the remote database 108.

If the user wishes to strike again, the method returns to initialize the IKMS 100, decision 326. Of course, for at least one embodiment, rapid succession strikes may utilize the same baseline data so as to not impose an additional wait time upon the user.

In light of the above model and review of the method 300 for determining impact kinetics, the following description provides a more refined embodiment for how such impact kinetics may be determined in accordance with at least one embodiment of the present invention.

As shown in FIG. 4B, the force 414 (of the strike) on the strike target 102 produces a moment of inertia, herein referred to as a moment (\vec{M}) 416 on the strike target 102 around the pivot point 126. A moment of inertia of an object, such as strike target 102, is a numerical value that can be calculated for any ridged body that is undergoing physical rotation about a vided axis—e.g., the strike target 102 about the Pivot attachment 126. This moment causes the angular acceleration of the strike target 102 around the pivot point 126. Using conservation of momentum in Equation 1:

−Σ\vec{M}=I\vec{α} Equation 1:

The only moment on the strike target 102 is due to the force applied by the impact and the strike target 102 mass once displaced, therefore Equation 2 is:

Σ\vec{M}=\vec{F}O̅T̅ Equation 2:

Equating results in Equation 3:

∴\vec{F}O̅T̅=I\vec{α} Equation 3:

The angular acceleration can be determined from the instantaneous linear acceleration measured by the accelerometer distance to the pivot point (Equation 4):

$$\vec{α} = \frac{\vec{a}}{\overline{OA}} \qquad Equation 4$$

The standard equation for the natural frequency of a compound pendulum may be applied to the strike target 102 in Equation 5.

$${ω}_{n} = \sqrt{\frac{mg \overline{OC}}{{I}_{P}}} \qquad Equation 5$$

In this formula:

ωₙ the circular frequency of the compound pendulum in rad/s.

I_{P} the moment of inertia of the compound pendulum in kgm².

The circular frequency is determined in Equation 6 from the frequency (f).

−ωₙ=2πf Equation 6:

Combining these two equations gives Equation 7:

$$∴ {I}_{P} = \frac{mg \overline{OC}}{{( 2 π f )}^{2}} \qquad Equation 7$$

The force can now be calculated by combining Equations 1, 2, and 3 to produce

$$∴ \vec{F} = \frac{mg \overline{OC} {\vec{a}}_{Tan}}{{\overline{OT} ( 2 π f )}^{2} \overline{OA}} \qquad Equation 8$$

The tangential component of \vec{a} will be used as the strike target 102 pivots primarily around the horizontal axis and any radial stretching of the strike target 102 will be neglected. That means \vec{a} will be replaced by \vec{a}_{Tan}.

$$– ∴ {\vec{F}}_{Tan .} = \frac{mg \overline{OC} {\vec{a}}_{Tan}}{{\overline{OT} ( 2 π f )}^{2} \overline{OA}} \qquad Equation 9$$

The above equation will give the instantaneous impact force as a function of instantaneous acceleration as recorded by the accelerometer during the period of impact. In Equation 9, mass (m) is the weight of the strike target 102. f is the frequency of the strike target 102 determined by oscillation of the strike target 102. C is the center of gravity and A is the accelerometer position, attached to the back surface of the strike target 102, directly opposite to the impact target. This specific position was chosen because it was found from preliminary tests that other positions (i.e. top, bottom, and next to the target signal) resulted in significant signal noise due to transverse vibration of the strike target 102 during impact.

Following Equation 9, the value of \vec{a}_{Tan}needs to be found from the accelerometer signal. As the accelerometer is mounted in a specific position, but arbitrary orientation, it is first necessary to determine the orientation of the accelerometer. This is done by measuring the three components of acceleration from the triaxial accelerometer when the strike target 102 is stationary (i.e. before impact). This signal (when stationary) comprises three components designated bₓ, b_{y}, b_{z} the combination of which produce the resultant vertical acceleration of gravity (g=9.81 m/s²). This is used as a calibration of magnitude, as well as orientation in Equation 10:

|b|=\sqrt{bₓ²+b_{y}²+b_{z}²} Equation 10:

In this formula:

|b| is the magnitude of vector \vec{b}.

To calibrate, Ψ is a correction factor applied to all signals to ensure that |b| equals exactly 1 g. Calculated in Equation 11:

$$g = Ψ \vert b \vert \\ ∴ Ψ = \frac{g}{\vert b \vert} \qquad Equation 11$$

During impact, it is necessary to remove the force of gravity from the accelerometer data. The triaxial accelerometer signal during impact is designated as \vec{a} where:

−|a|=\sqrt{aₓ²+a_{y}²+a_{z}²} Equation 12:

The angle between \vec{a} and \vec{b} also needs to be determined in order to remove gravity through Equation 13. The angle between the two vectors is:

$$θ = {cos}^{- 1} \frac{\vec{a} \cdot \vec{b}}{\vert a \vert \vert b \vert} \qquad Equation 13$$

This is the angle between \vec{a} and the vertical.

Where from the standard vector calculation is Equation 14:

\vec{a}·\vec{b}=aₓbₓ+a_{y}b_{y}+a_{z}b_{z} Equation 14:

With the angle of the accelerometer found, the tangential acceleration can be calculated in Equation 15 and the variables can be seen in FIG. 4B:

\vec{a}_{Tan}=\sqrt{(||Ψ cos θ−|b|Ψ)²+(|a|Ψ sin θ)²} Equation 15:

Equations 4 and 5 were combined, and together with the recording of the accelerometer signal, the instantaneous kinetics were calculated during the impact (peak and mean Force). From this force versus time trace, other parameters are determined, and most specifically, Impulse.

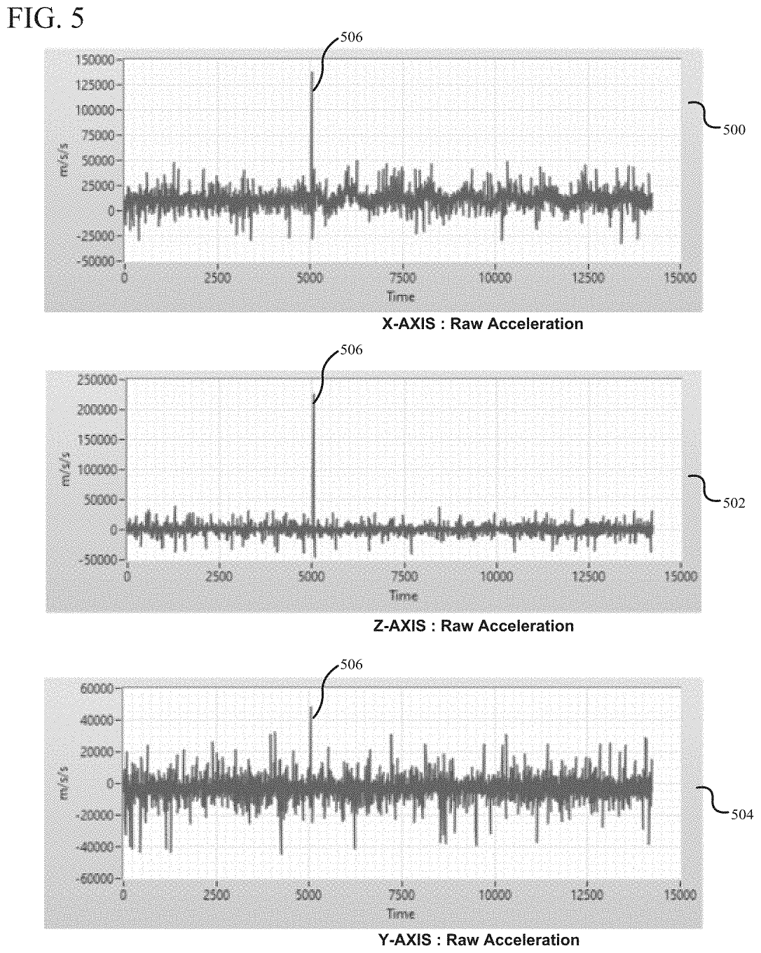

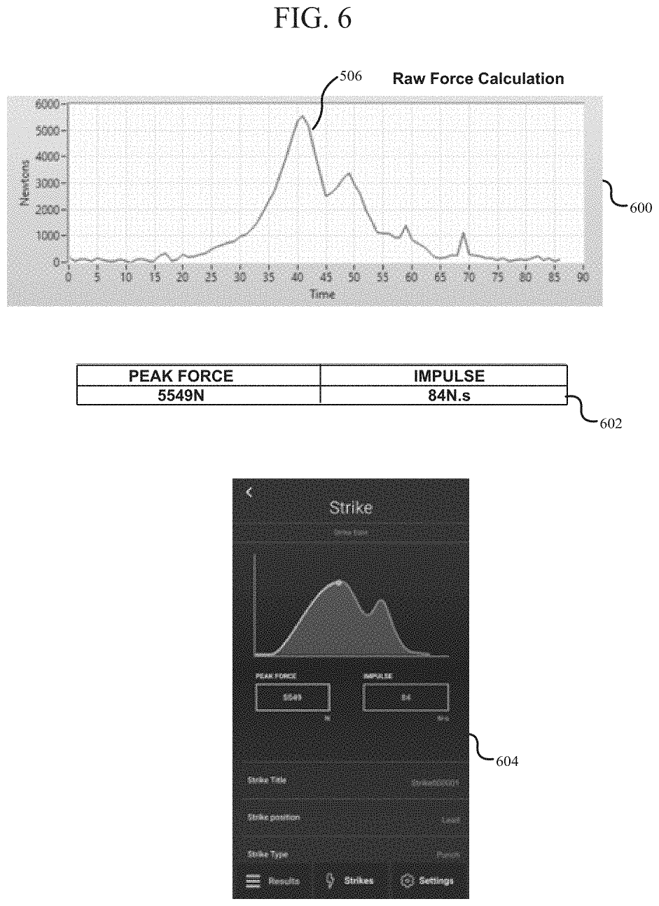

With respect to the above discussion of method 300 and Equations 1-9, FIGS. 5 and 6 present example data from an operating embodiment of IKMS 100. More specifically, FIGS. 5 and 6 present an overview of the steps for the flow of data within at least one embodiment of IKMS 100. As shown in FIG. 5 IKMS 100 captures raw acceleration data—graph 500 showing the X-Axis, graph 502 showing the Z-Axis, and graph 504 showing the Y-axis. As will be appreciated, before and after the strike 506, due to the sensitivity of the triaxial accelerometer there is baseline noise in each graph due to any number of environmental factors—wind, ground or building vibration, etc. . . .

As is understood from a review of graphs 500, 502 and 504, upon a strike to the strike target 102, the acceleration in each axis is different. These different acceleration values are aggregated in the raw force curve calculation shown in graph 600. From the data yielding graph 600, IKMS 100 determines the Peak Force to be 5549N, which is consistent with graph 600. As Impulse is the integration of force over time, IKMS 100 further determines that the Impulse for the strike is 84 N·s, shown in table 602.

For at least one embodiment these values are provided to a user 112 by way of his or her first device 114. More specifically, for at least one embodiment where the user 112 has a smart phone as their first device 114, the application 106 provides an easy to view visual graph 604, with peak force and impulse. The user 112 may further optionally supply additional information, such as a title for the recorded strike, the strike position, and strike type. The application 106 may further provide options for summary views of results, specific results for strikes, as well as general settings options.

Indeed, with substantially immediate feedback with visual representation of the strike, the user 112 can relate the information provided by IKMS 100 to how they performed and recall the strike, and adjust accordingly for improvement or refinement.

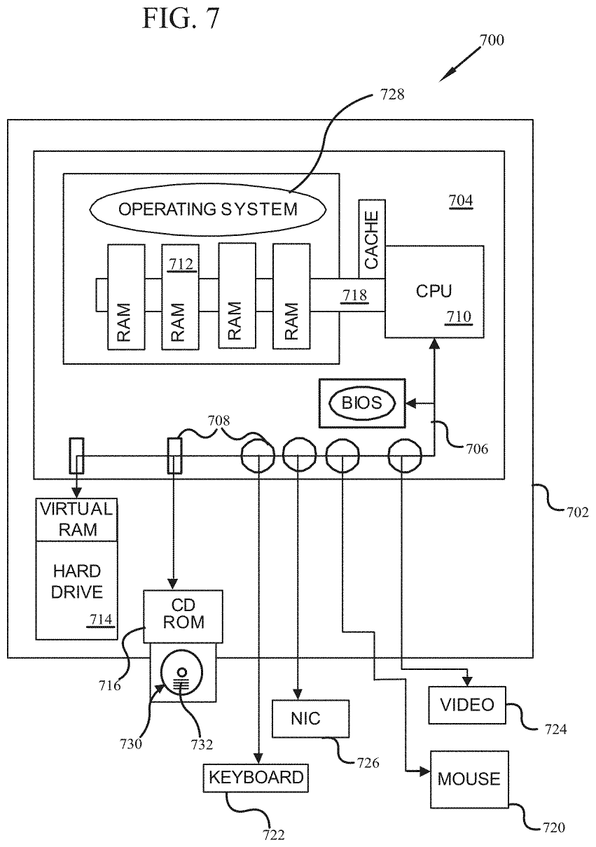

To expand upon the initial suggestion of at least the detection system 104, the database 108, the first device 114 and other systems comprising IKMS 100 being computer systems adapted to their specific roles, FIG. 7 is a high level block diagram of an exemplary computer system 700 such as may be provided for one or more of the elements comprising at least the detection system 104, the database 108, the first device 114 whether provided as distinct individual systems or integrated together in one or more computer systems.

Computer system 700 has a case 702, enclosing a main board 704. The main board 704 has a system bus 706, connection ports 708, a processing unit, such as Central Processing Unit (CPU) 710 with at least one microprocessor (not shown) and a memory storage device, such as main memory 712, hard drive 714 and CD/DVD ROM drive 716.

Memory bus 718 couples main memory 712 to the CPU 710. A system bus 706 couples the hard disc drive 714, CD/DVD ROM drive 716 and connection ports 708 to the CPU 710. Multiple input devices may be provided, such as, for example, a mouse 720 and keyboard 722. Multiple output devices may also be provided, such as, for example, a video monitor 724 and a printer (not shown). As computer system 700 is intended to be interconnected with other computer systems in the IKMS 100 a combined input/output device such as at least one network interface card, or NIC 726 is also provided.

Computer system 700 may be a commercially available system, such as a desktop workstation unit provided by IBM®, Dell Computers®, Gateway®, Apple®, or other computer system provider, or adapted computer system as provided for a mobile computing device such as an iPhone® or Android® device, which may have different peripheral components, or lack peripherals entirely, such that they are somewhat different from the exemplary system 700 shown for ease of illustration and discussion. Computer system 700 may also be a networked computer system, wherein memory storage components such as hard drive 714, additional CPUs 710 and output devices such as printers are provided by physically separate computer systems commonly connected together in the network.

Those skilled in the art will understand and appreciate that the physical composition of components and component interconnections are comprised by the computer system 700, and select a computer system 700 suitable for one or more of the computer systems incorporated in the formation and operation of IKMS 100.

When computer system 700 is activated, preferably an operating system 728 will load into main memory 712 as part of the boot strap startup sequence and ready the computer system 700 for operation. At the simplest level, and in the most general sense, the tasks of an operating system fall into specific categories, such as, process management, device management (including application and user interface management) and memory management, for example. The form of the computer-readable medium 730 and language of the program 732 are understood to be appropriate for and functionally cooperate with the computer system 700.

Moreover, variations of computer system 700 may be adapted to provide the physical elements of one or more components comprising each at least detection system 104, the database 108, the first device 114, the switches, routers and such other components as may be desired and appropriate for the methods and systems for determining an appropriate dose of a product as set forth above.

Changes may be made in the above methods, systems and structures without departing from the scope hereof. It should thus be noted, that the matter contained in the above description and/or shown in the accompanying drawings should be interpreted as illustrative and not in a limiting sense. Indeed, many other embodiments are feasible and possible, as will be evident to one of ordinary skill in the art. The claims that follow are not limited by or to the embodiments discussed herein, but are limited solely by their terms and the Doctrine of Equivalents.

What is claimed:

1. A system for measuring impact kinetics, comprising:
a strike target having an outer surface with at least one pressure sensor proximate thereto, a known center of gravity and a known weight, the strike target further having a pivot attachment to an overhead support;
at least one accelerometer associated with the strike target;
a detection system in communication with the at least one pressure sensor and the accelerometer, the detection system initiating kinetic impact determination upon a signal to evaluate a strike, the strike producing strike data including at least a moment of the bag around the pivot attachment, this moment causing an angular acceleration (\vec{α}) of the strike target around the pivot as detected by the at least one accelerometer and reported to the detection system;
wherein the detection system determines impact kinetics as a natural frequency (ωₙ) and a moment of inertia (I_{P}) of the strike target as a compound pendulum, determines the circular frequency (−ωₙ) of the compound pendulum, and a resulting impact force as a function of instantaneous acceleration, the impact kinetics reported to a user.

2. The system of claim 1, wherein the orientation of the accelerometer is determined by measuring three components of acceleration when the strike target is stationary (bₓ, b_{y}, b_{z}) to produce a resultant vertical acceleration of gravity (g=9.81 m/s²), as an initial calibration of magnitude (|b|).

3. The system of claim 2, wherein (|b|) is the magnitude of vector \vec{b}.

4. The system of claim 3, wherein a correction factor (Ψ) is calculated by
$$Ψ = \frac{g}{\vert b \vert} .$$

5. The system of claim 3, wherein removal of gravity from the accelerometer data is performed by;
determining a magnitude of vector \vec{a}, in accordance with −|a|=\sqrt{a}ₓ²+a_{y}²+a_{z}²; and
determining an angle (θ) between \vec{a} and \vec{b} as
$$θ = {cos}^{- 1} \frac{\vec{a} \cdot \vec{b}}{\vert a \vert \vert b \vert} .$$

6. The system of claim 5, wherein tangential acceleration of the accelerometer is determined by \vec{a}_{Tan}=\sqrt{(|a|Ψ cos θ−|b|Ψ)²+(|a|Ψ sin θ)²}.

7. The system of claim 1, wherein the strike target is a tear drop punching bag.

8. The system of claim 1, wherein the strike target is a small punching bag.

9. The system of claim 1, wherein the at least one accelerometer is a triaxial accelerometer.

10. The system of claim 1, wherein there are at least three accelerometer, an x-axis accelerometer, a y-axis accelerometer and a z-axis accelerometer.

11. The system of claim 1, wherein the at least one accelerometer associated with the strike target is disposed within the strike target.

12. The system of claim 1, wherein the at least one accelerometer associated with the strike target is disposed upon the surface of the strike target.

13. A method for measuring impact kinetics, comprising:
providing a strike target having a pivot attachment to an overhead support, an outer surface with at least one pressure sensor proximate thereto, and a known center of gravity, and weight of the bag, the strike target having at least one accelerometer associated therewith;
providing a detection system in communication with the at least one pressure sensor and the accelerometer; and
initiating kinetic impact determination upon a signal to evaluate a strike, the strike producing strike data including at least a moment of the bag around the pivot attachment, this moment causing an angular acceleration (\vec{α}) of the target around the pivot as detected by the at least one accelerometer and reported to the detection system;
wherein the detection system determines impact kinetics as a natural frequency (ωₙ) and a moment of inertia (I_{P}) of the strike target as a compound pendulum, determines the circular frequency (−ωₙ) of the compound pendulum, and a resulting impact force as a function of instantaneous acceleration, the impact kinetics reported to a user.

14. The method of claim 13, wherein the orientation of the accelerometer is determined by measuring three components of acceleration when the strike target is stationary (bₓ,b_{y},b_{z}) to produce a resultant vertical acceleration of gravity (g=9.81 m/s²), as an initial calibration of magnitude (|b|).

15. The method of claim 14, wherein (|b|) is the magnitude of vector \vec{b}.

16. The method of claim 15, wherein a correction factor (Ψ) is calculated by
$$Ψ = \frac{g}{\vert b \vert} .$$

17. The method of claim 15, wherein removal of gravity from the accelerometer data is performed by;
determining a magnitude of vector \vec{a}, in accordance with −|a|=\sqrt{aₓ²+a_{y}²+a_{z}²}; and
determining an angle (θ) between \vec{a} and \vec{b} as
$$θ = {cos}^{- 1} \frac{\vec{a} \cdot \vec{b}}{\vert a \vert \vert b \vert} .$$

18. The method of claim 18, wherein tangential acceleration of the accelerometer is determined by \vec{a}_{Tan}=\sqrt{(|a|Ψ cos θ−|b|Ψ)²+(|a|Ψ sin θ)²}.

19. The method of claim 13, wherein the strike target is a tear drop punching bag.

20. The method of claim 13, wherein the strike target is a small punching bag.

21. The method of claim 13, wherein the at least one accelerometer is a triaxial accelerometer.

22. The method of claim 13, wherein there are at least three accelerometer, an x-axis accelerometer, a y-axis accelerometer and a z-axis accelerometer.

23. The method of claim 13, wherein the signal is provided by the pressure sensor indicating a user has struck the strike target.

24. The method of claim 13, wherein the signal is an initialization signal indicating that the strike target is at rest so as to determine initial baseline values to determine impact kinetics upon a strike to the strike target.

25. A system for measuring impact kinetics, comprising:
A strike detection system having at least one processor and adapted to receive strike data from a strike target, the strike data provided by at least one pressure sensor and at least one accelerometer associated with the strike target having a known center of gravity and a known weight, the strike target further having a pivot attachment to an overhead support;
at least one remote strike detection controller having at least one processor and non-volatile memory coupled to the processor having processor executable instructions to direct operation of strike detection system, the strike detection controller having a wireless network component coupled to the processor and the non-volatile memory and in communication with the strike detection system;
a strike evaluator for initiating kinetic impact determination upon a signal to evaluate a strike, the strike producing the strike data as a moment of the bag around the pivot attachment, this moment causing an angular acceleration (\vec{α}) of the strike target around the pivot as detected by the at least one accelerometer and reported to the detection system;
wherein the strike evaluator determines impact kinetics as a natural frequency (ωₙ) and a moment of inertia (I_{P}) of the strike target as a compound pendulum, determines the circular frequency (−ωₙ) of the compound pendulum, and a resulting impact force as a function of instantaneous acceleration, the impact kinetics reported to a user.

26. The system of claim 25, wherein the strike evaluator is a component of the strike detection system

27. The system of claim 25, wherein the strike evaluator is a component of the strike detection controller.

28. The system of claim 25, wherein the strike detection controller is a user computing device adapted by executable instructions provided as an application to adapt the user computing device as a remote strike detection controller.

29. The system of claim 25, further including a remote database system in network communication with the strike detection system and the strike detection controller, the remote database having a user account for each user known to the system, the database further recording the strike data associated with each user.

30. The system of claim 29, wherein the strike evaluator is a component of the database system.

31. The system of claim 25, wherein the orientation of the accelerometer is determined by measuring three components of acceleration when the strike target is stationary (bₓ, b_{y}, b_{z}) to produce a resultant vertical acceleration of gravity (g=9.81 m/s²), as an initial calibration of magnitude (|b|).

32. The system of claim 31, wherein (|b|) is the magnitude of vector \vec{b}.

33. The system of claim 32, wherein a correction factor (Ψ) is calculated by
$$Ψ = \frac{g}{\vert b \vert} .$$

34. The system of claim 32, wherein removal of gravity from the accelerometer data is performed by;
determining a magnitude of vector \vec{a}, in accordance with −|a|=\sqrt{aₓ²+a_{y}²+a_{z}²}; and
determining an angle (θ) between \vec{a} and \vec{b} as
$$θ = {cos}^{- 1} \frac{\vec{a} \cdot \vec{b}}{\vert a \vert \vert b \vert} .$$

35. The system of claim 34, wherein tangential acceleration of the accelerometer is determined by \vec{a}_{Tan}=\sqrt{(|a|Ψ cos θ−|b|Ψ)²+(|a|Ψ sin θ)²}.

36. The system of claim 25, wherein the strike target is a tear drop punching bag.

37. The system of claim 25, wherein the strike target is a small punching bag.

38. The system of claim 25, wherein the at least one accelerometer is an triaxial accelerometer.

39. The system of claim 25, wherein there are at least three accelerometers, an x-axis accelerometer, a y-axis accelerometer and a z-axis accelerometer.

40. The system of claim 25, wherein the at least one accelerometer associated with the strike target is disposed within the strike target.

41. The system of claim 25, wherein the at least one accelerometer associated with the strike target is disposed upon the surface of the strike target.
